(19) United States
(12) Patent Application Publication
(10) Pub. No.: US 2017/0105793 A1
(43) Pub. Date: Apr. 20, 2017

(54) ENERGY DELIVERY DEVICES AND RELATED METHODS OF USE

(71) Applicant: Boston Scientific Scimed, Inc., Maple Grove, MN (US)

(72) Inventors: Hong CAO, Maple Grove, MN (US); Bruce R. FORSYTH, Hanover, MN (US)

(73) Assignee: Boston Scientific Scimed, Inc.

(21) Appl. No.: 15/290,580

(22) Filed: Oct. 11, 2016

Related U.S. Application Data
(60) Provisional application No. 62/241,961, filed on Oct. 15, 2015.

(51) Int. Cl. A61B 18/14; A61N 1/32; A61N 1/30; A61B 18/12

(52) U.S. Cl. CPC A61B 18/1492; A61B 18/1233; A61N 1/327; A61N 1/30; A61N 1/325; A61B 2018/0022

(57) ABSTRACT

An energy delivery system may include an expandable member having a plurality of electrodes, including pairs of adjacent electrodes, and a generator configured to supply an electric voltage to each of the plurality of electrodes. The energy delivery system also may include a controller coupled to the plurality of electrodes and the generator. The controller may be configured to measure an impedance of tissue disposed between the electrodes of each pair of adjacent electrodes, and determine, based on the measured impedances, whether cancerous tissue is disposed between any of the electrodes of each pair of adjacent electrodes. The controller also may be configured to apply a voltage between the electrodes of each pair of adjacent electrodes determined to have cancerous tissue disposed between them.

### CROSS-REFERENCE TO RELATED APPLICATION(S)

This patent application claims the benefit under 35 U.S.C. §119 to U.S. Provisional Patent Application No. 62/241,961, filed on Oct. 15, 2015, the entirety of which is incorporated herein by reference.

### TECHNICAL FIELD

Examples of the present disclosure relate generally to energy delivery devices and related methods of use. More particularly, examples of the present disclosure relate to devices and methods for treating tissue (e.g., cancerous or soft) with electric fields.

### BACKGROUND

Various thermal treatments have been utilized to damage cancerous tissues, such as, e.g., tumors. While thermal treatments may be therapeutically effective, complications such as tissue charring may occur. Additionally, thermal treatments may be complicated because of a heat sink effect of blood flow through an artery or vein near the treatment site. That is, flowing blood may carry heat delivered from a delivery device away from the treatment site, reducing the efficiency of various treatments.

Irreversible electroporation is a non-thermal procedure that can be used to treat cancer or soft tissues. However, conventional electroporation therapies utilize needles in complex percutaneous or open-access procedures. Further, the electroporation needles are unable to get close to various treatment sites such as, e.g., bile ducts or hepatic arteries.

### SUMMARY OF THE DISCLOSURE

In one aspect, the present disclosure is directed to an energy delivery system. The energy delivery system may include an expandable member having a plurality of electrodes, including pairs of adjacent electrodes, and a generator configured to supply an electric voltage to each of the plurality of electrodes. The energy delivery system also may include a controller coupled to the plurality of electrodes and the generator. The controller may be configured to measure an impedance of tissue disposed between the electrodes of each pair of adjacent electrodes, and determine, based on the measured impedances, whether cancerous tissue is disposed between any of the electrodes of each pair of adjacent electrodes. The controller also may be configured to apply a voltage between the electrodes of each pair of adjacent electrodes determined to have cancerous tissue disposed between them.

Each electrode of the plurality of electrodes may be circumferentially adjacent to one or more electrodes and longitudinally adjacent to one or more electrodes. The expandable member may include a plurality of rows of electrodes, wherein each row of electrodes includes a plurality of longitudinally spaced electrodes, wherein the plurality of rows are circumferentially spaced from one another. All adjacent and circumferentially spaced electrodes may be spaced apart from one another by the same circumferential distance. All adjacent and longitudinally spaced electrodes may be spaced apart from one another by the same longitudinal distance. The expandable member may be a balloon, and the plurality of electrodes may be disposed on or adjacent to an outer surface of the balloon. At least some of the plurality of electrodes may include flexible circuits attached to an outer surface of the expandable member. The expandable member may be an expandable basket having a plurality of legs, and each leg of the plurality of legs may include a plurality of longitudinally spaced electrodes. The energy delivery system may include an actuating member that extends from a proximal end of the expandable member, through a volume defined by the plurality of legs, to a distal end of the expandable member. The plurality of electrodes may be coupled to opposite poles of the generator. Adjacent electrodes of the plurality of electrodes may be configured to deliver energy of opposite polarity. The controller may determine that cancerous tissue is located between a pair of circumferentially adjacent electrodes or a pair of longitudinally adjacent electrodes if the measured impedance between the pair of circumferentially adjacent electrodes or the pair of longitudinally adjacent electrodes is smaller than a threshold impedance value. The generator may be configured to supply voltage to each of the electrodes in a manner sufficient to cause electroporation of tissue. The generator may be configured to create an electrical field strength within tissue from 250 to 3000 volt/cm. The generator may be configured to deliver pulses with a pulse width from 1 ns to 1000 μs and a pulse interval from 10 ms to 100 s.

In another aspect, the present disclosure is directed to an energy delivery system. The energy delivery system may include an expandable balloon, and a plurality of electrodes disposed on an outer surface of the balloon, and each electrode of the plurality of electrodes may be circumferentially adjacent to one or more electrodes and longitudinally adjacent to one or more electrodes. The energy delivery system also may include a generator configured to supply an electric voltage to each of the plurality of electrodes, wherein any two circumferentially adjacent electrodes may be coupled to opposite poles of the generator, and wherein any two longitudinally adjacent electrodes may be coupled to opposite poles of the generator, wherein the generator is configured to supply the electric voltage in a manner sufficient to cause electroporation of tissue. The energy delivery system also may include a controller coupled to the plurality of electrodes and the generator. The controller may be configured to measure an impedance of tissue disposed between pairs of circumferentially adjacent electrodes and pairs of longitudinally adjacent electrodes, and determine, based on the measured impedances, whether cancerous tissue is disposed between any pairs of circumferentially adjacent electrodes and any pairs of longitudinally adjacent electrodes. The controller also may be configured to apply a voltage only between any pairs of circumferentially adjacent electrodes and any pairs of longitudinally adjacent electrodes determined to have cancerous tissues disposed between them, wherein the controller may determine that cancerous tissue is located between a pair of circumferentially adjacent electrodes or a pair of longitudinally adjacent electrodes if the measured impedance between the pair of circumferentially adjacent electrodes or the pair of longitudinally adjacent electrodes is smaller than a threshold impedance value.

The expandable member may include a plurality of rows of electrodes, wherein each row of electrodes may include a plurality of longitudinally spaced electrodes, wherein each electrode of the plurality of electrodes may be positioned within one of the plurality of rows, and may lie in a single plane normal to a longitudinal axis of the expandable member with a correspondingly positioned electrode from each other row.

In yet another aspect, the present disclosure is directed to a method of treating tissue adjacent a body lumen. The method may include measuring an impedance of tissue disposed between pairs of adjacent electrodes of an energy delivery device; applying an electric field only between pairs of adjacent electrodes determined to have cancerous tissue disposed between them, wherein a controller may determine that cancerous tissue is disposed between a given pair of adjacent electrodes based on the measured impedance between the given pair of adjacent electrodes, wherein the electric field causes electroporation of the tissue disposed within the electric field.

The body lumen may be a bile duct, a lung airway, a hepatic artery, or a lumen of a liver or pancreas. The electric field may be a bipolar electric field.

Additional objects and advantages of the disclosure will be set forth in part in the description which follows, and in part will be obvious from the description, or may be learned by practice of the disclosure. The objects and advantages of the disclosure will be realized and attained by means of the elements and combinations particularly pointed out in the appended claims.

The accompanying drawings, which are incorporated in and constitute a part of this specification, illustrate several examples of the present disclosure and together with the description, serve to explain the principles of the invention.

### BRIEF DESCRIPTION OF THE DRAWINGS

FIG. 1 is a schematic view of a system for delivering energy to tissue surrounding a body lumen according to one example of the present disclosure.

FIG. 2 is a cross-sectional view of an energy delivery device configuration according to one example of the present disclosure.

FIG. 3 is a cross-sectional view of an energy delivery device configuration according to another example of the present disclosure.

FIGS. 4-6 are side views of energy delivery devices, according to additional examples of the present disclosure.

FIG. 7 is a cross-sectional view of an energy delivery device disposed within a body lumen, according to an example of the present disclosure.

### DESCRIPTION OF THE EXAMPLES

Reference will now be made in detail to examples of the present disclosure, which are illustrated in the accompanying drawings. Wherever possible, the same reference numbers will be used throughout the drawings to refer to the same or like parts.

Energy Delivery Devices

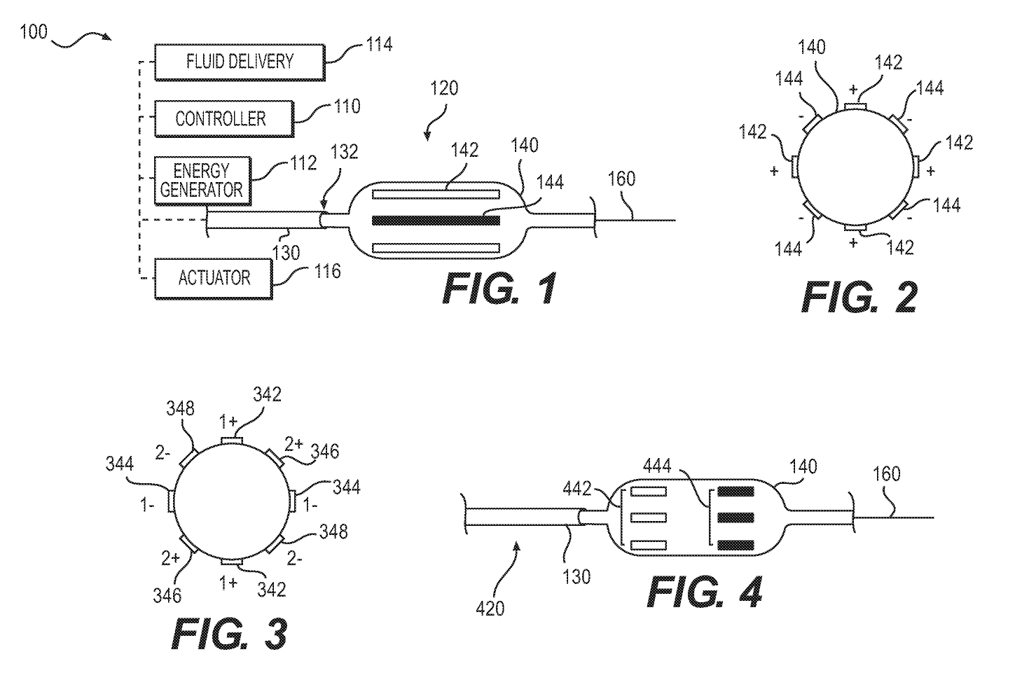

Examples of the present disclosure relate to devices and methods for controlling the application of energy to tissue surrounding or otherwise adjacent to a body lumen of a patient. FIG. 1 illustrates a system 100 for delivering energy, in accordance with a first example of the present disclosure. The system may include a controller 110, an energy generator 112, a fluid delivery system 114, an actuator 116, and an energy delivery device 120.

Controller 110 may be operatively coupled to energy generator 112, fluid delivery system 114, actuator 116, and energy delivery device 120. Controller 110 may be configured to optimize energy delivery to a patient based on algorithms and/or inputs from one or more sensing devices. In some examples, the controller 110 may include a processor that is generally configured to accept information from the system and system components, and process the information according to various algorithms to produce control signals for controlling energy generator 112, fluid delivery system 114, and energy delivery device 120. The processor may accept information from the system and system components, process the information according to various algorithms, and produce information signals that may be directed to visual indicators, digital displays, audio tone generators, or other indicators of, e.g., a user interface, in order to inform a user of the system status, component status, procedure status or any other useful information that is being monitored by the system. The processor may be a digital IC processor, analog processor or any other suitable logic or control system that carries out the control algorithms. In some examples, controller 110 may record treatment parameters such as, e.g., voltage, current, impedance and other suitable treatment parameters so that they may be accessed for concurrent or subsequent analysis.

Energy generator 112 may be configured to apply voltages across electrodes to produce electric fields. Energy generator 112 may be adapted, for example, to promote electrically assisted therapeutic agent delivery within a subject, including electroporation. Power sources and power application schemes for use in electroporation are known in the medical device art. Energy generator 112 may be configured to deliver irreversible or reversible electroporation therapies via electrodes disposed within a body lumen. Irreversible electroporation may be an electrical method of causing cell death by apoptosis. In some examples, there may be 10 to 100 pulses per treatment, with a pulse length of 1 millisecond to 1 microsecond. There may be 100 to 1000 milliseconds between pulses, with a field strength from 250 to 3000 volt/cm. Pulses may be configured with a pulse width from 1 ns to 1000 μs and pulse interval from 10 ms to 100 s, and single or multiple pulse trains. Pulse timing, or pauses between pulse trains (e.g., 1 to 100 second delays between n=50 pulses) may improve cell destruction. Voltages applied at this level may cause muscle contractions. Drugs or other agents may be administered to a patient to avoid these muscle contractions. It is contemplated that energy generator 112 may be operated using any other suitable parameter. In some examples energy generator 112 or controller 110 may monitor ECG signals to synchronize the electroporation pulse at optimal points (e.g., on an R-wave or T-wave) during or just before the refractory period.

Fluid delivery system 114 may be a standard balloon inflation device or may include an inflation pump (not shown) that is in fluid communication with the energy delivery device 120. More specifically, activation of the pump by a user may cause the energy delivery device 120 to be selectively moved between a deflated configuration and an inflated configuration, for example, when energy delivery device 120 is an inflatable balloon.

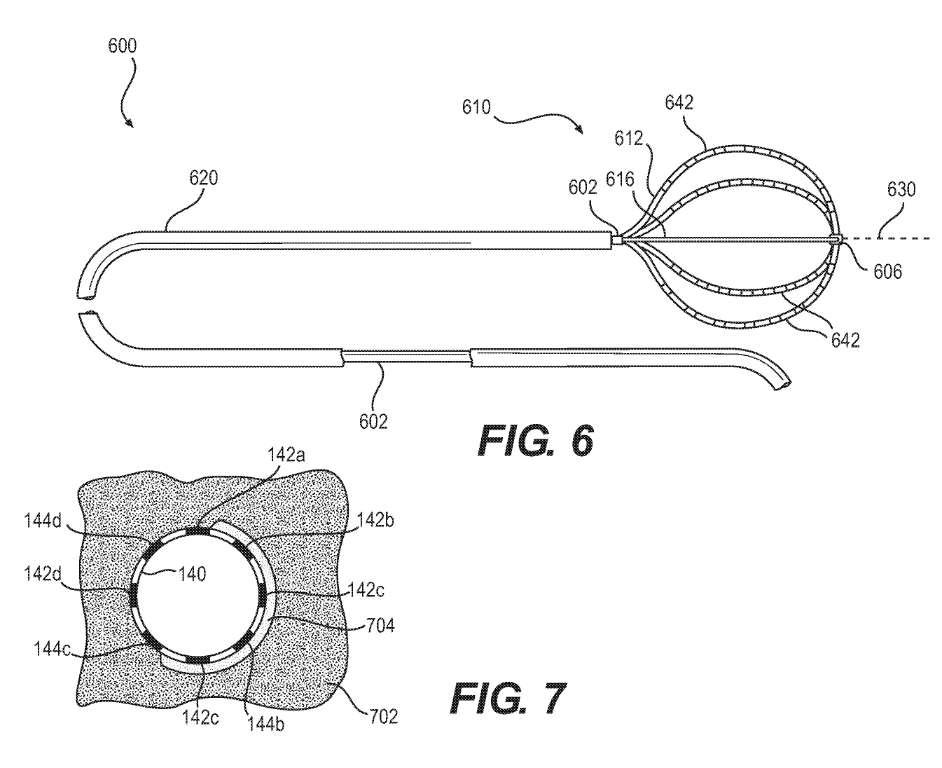

Actuator 116 may be any suitable automatic and/or user operated device in operative communication with energy generator 112 and/or energy delivery device 120 via a wired or wireless connection, such that actuator 116 may be configured to enable activation of energy generator 112 and/or energy delivery device 120. Actuator 116 may therefore include a switch, a push-button, computer or other suitable actuator configured to operate energy generator 112. Further, actuator 116 may include a handle, slider, trigger, and/or other suitable mechanism configured to apply a force to an actuating member (e.g., actuating member 616 of FIG. 6) of an energy delivery device.

Energy delivery device 120 may include an elongate member 130 having a proximal portion (not shown) and a distal portion 132. Elongate member 130 may be any suitable longitudinal device configured to be inserted into a cavity and/or passageway of a body. Elongate member 130 may further include any suitable stiff or flexible material configured to enable movement of energy delivery device 120 through a cavity and/or passageway in a body. In one example, elongate member 130 may be sufficiently flexible to enable elongate member 130 to conform to the lumen, cavity and/or passageway through which it is inserted.

Elongate member 130 may be any suitable size, shape, and or configuration such that elongate member 130 may be configured to pass through a lumen of an access device. The access device may be any suitable endoscopic member, such as, e.g., an endoscope, a ureteroscope, a colonoscope, a hysteroscope, a uteroscope, a bronchoscope, a cystoscope, a sheath, or a catheter. The access device may include one or more additional lumens configured for the passage of a variety of surgical equipment, including, but not limited to, imaging devices and tools for irrigation, vacuum suctioning, biopsies, and drug delivery.

Elongate member 130 may be solid or hollow. Similar to the access device, elongate member 130 may include one or more lumens substantially similar to those of the access device, including, for the circulation of inflation fluid. Elongate member 130 may further include an atraumatic exterior surface having a rounded shape and/or a coating. The coating be any coating known to those skilled in the art enabling ease of movement of energy delivery device 120 through the access device and a lumen and/or cavity within a body. The coating may therefore include, but is not limited to, a lubricious coating and/or an anesthetic.

An expandable member 140 may be disposed at distal end 132 of elongate member 130. The expandable member 140 may be a radially expandable balloon that is configured to be inflated with fluid supplied by fluid delivery system 114. The balloon may be formed of noncompliant polymer materials or semi-compliant materials. Semi-compliant balloons may include polyether-block-amide (PEBA) copolymers and nylon materials. Non-compliant balloons may include relatively rigid or stiff polymeric materials, such as, e.g., thermoplastic polymers, thermoset polymeric materials, poly(ethylene terephthalate), polyimide, thermoplastic polyimide, polyamides, polyesters, polycarbonates, polyphenylene sulfides, polypropylene and rigid polyurethanes. Non-compliant balloons may be inflated only to a fixed diameter once they have been inflated to a minimum threshold pressure.

The energy delivery device 120 may be used in combination with a catheter guide element such as a guide wire 160. In use, both the guide wire 160 and the elongate member 130 may be fed through and guided to a treatment site by, e.g., a guide catheter (not shown). Energy delivery device 120 may thus include a separate guide wire lumen (not shown) that extends through at least expandable member 140.

One or more electrodes 142, 144 may be disposed on an outer surface of expandable member 140. Electrodes 142 and 144 may be configured to deliver and receive electric voltage from other electrodes to produce electric fields in the body. In some examples, electrodes 142 and 144 may be flexible circuits formed of a base polymer material having a metal or other suitable conductive material disposed on the base polymer material. The conductive material may be gold, platinum, stainless steel, cobalt alloys, and other non-oxidizing materials. The flexible circuit may include copper wires which may be lithographically printed thereon. While each electrode 142 and 144 is shown as a single, longitudinally extending electrode, it is contemplated that electrodes 142 and 144 may be include a plurality of smaller electrodes that are spaced longitudinally from one another along a longitudinal axis of expandable member 140. When electrodes 142 and 144 include flexible circuits, the flexible circuits may be coupled to an outer surface of expandable member 140 via an adhesive bond. That is, adhesive may be applied to the flexible circuit and/or to the outer surface of expandable member 140.

In another example, electrodes 142 and 144 may include a conductive ink. A suitable conductive ink can be composed of a binder or base, and a conductive filler dispersed in the binder. The binder can be composed of a flexible, compliant polymer (e.g., urethane), silicone, or another suitable biocompatible material configured to stretch with the expandable member 140 during device operation. The conductive filler may include particles that have a variety of different sizes, shapes, distributions, and/or concentrations within the binder. For example, the conductive filler may include both small and large conductive flakes. The conductive filler also may include conductive fibers, strands, spheres, rods, cylinders, strips, pellets, or combinations thereof. The individual particles or fibers of the conductive filler may be oriented to slide over one another (e.g., overlapping) to maintain contact as the corresponding conductive ink stretches and expands during operation so as to ensure conductive continuity and durability. The conductive filler may include silver, gold, copper, carbon, or other suitable biocompatible conductive materials.

Electrodes 142 and 144 may be circumferentially spaced from one another about the outer surface of expandable member 140. In some examples, the spacing of electrodes 142 and 144 may be uniform about the circumference of expandable member 140, although irregular or non-uniform spacing is also contemplated. Electrodes 142 may alternate with electrodes 144 about the circumference of expandable member 140. In some examples, electrodes 142 may be coupled to a first pole of energy generator 112, while electrodes 144 may be coupled to a second pole of energy generator 112 that is opposite of the first pole. Thus, energy delivery device 120 may be configured as a bipolar device whereby energy flows from one or more electrodes of the first pole, through tissues surrounding or otherwise adjacent to a body lumen, to one or more electrodes of the second pole. In other examples, energy delivery device 120 may be a monopolar device and electrodes 142 and 144 may be substantially similar to one another and may deliver energy through tissue surrounding the body lumen to a ground pad placed on the patient's skin, for example.

FIG. 2 is a cross-sectional view of expandable member 140 showing a first configuration of electrodes. In the example of FIG. 2, electrodes 142 may be configured to deliver electrical field energy of a first pole (+), while electrodes 144 are configured to deliver electrical field energy of a second pole (−). In some examples, all of electrodes 142 and 144 may be simultaneously activated, while in other examples, only some of electrodes 142 and 144 may be operated at a given time, or during a given treatment procedure as set forth below.

FIG. 3 shows an expandable member having an alternative configuration of electrodes. In the example of FIG. 3, the electrodes disposed around the expandable member 140 are configured to be activated in separate groups. For example, a first group of electrodes that includes electrodes 342 and 344 may be activated simultaneously. In this example, electrodes 342 and 344 may alternate with one another about the circumference of expandable member 140, and may be configured for bipolar energy delivery in a substantially similar manner as electrodes 142 and 144 set forth above. That is, electrodes 342 may be configured to deliver electric field energy of a first pole, while electrodes 344 may be configured to deliver electric field energy of a second pole that is different than the first pole. In the example of FIG. 3, electrodes 342 and 344 may be spaced further apart from one another than electrodes 142 and 144 of FIG. 2, and thus, may be configured to generate larger and deeper electrical fields, penetrating larger and deeper lesions.

A second group of electrodes including electrodes 346 and 348 may be configured to be activated separately from the first group of electrodes. The second group of electrodes may be substantially similar to the first group of electrodes, and may operate in a similar manner. Further, electrodes from the first group of electrodes (e.g., electrodes 342 and 344) may alternate with electrodes from the second group of electrodes (e.g., electrodes 346 and 348) about the circumference of expandable member 140, and may be configured for bipolar energy delivery in a substantially similar manner as electrodes 142 and 144 set forth above. That is, electrodes 346 may be configured to deliver electric field energy of a first pole, while electrodes 348 may be configured to deliver electric field energy of a second pole that is different than the first pole.

An energy delivery device 420 is shown in FIG. 4. Energy delivery device 420 may be substantially similar to energy delivery device 120 described above, except that energy delivery device 420 may include a first set of electrodes 442 and a second set of electrodes 444 that are longitudinally spaced from one another on the outer surface of expandable member 140. That is, the first set of electrodes 442 may be disposed proximally of the second set of electrodes 444. The first set of electrodes 442 may include a plurality of electrodes that are circumferentially spaced apart from one another about the circumference of the expandable member 140. Similarly, the second set of electrodes 444 may include a plurality of electrodes that are spaced apart from one another about the circumference of the expandable member 140. In one example, the first set of electrodes 442 may be configured to provide electric field energy of a first pole, while the second set of electrodes 444 may be configured to provide electric field energy of a second pole that is different than the first pole. Thus, energy delivery device 420 may be configured to create bipolar electric fields extending along various circumferentially-spaced longitudinal trajectories.

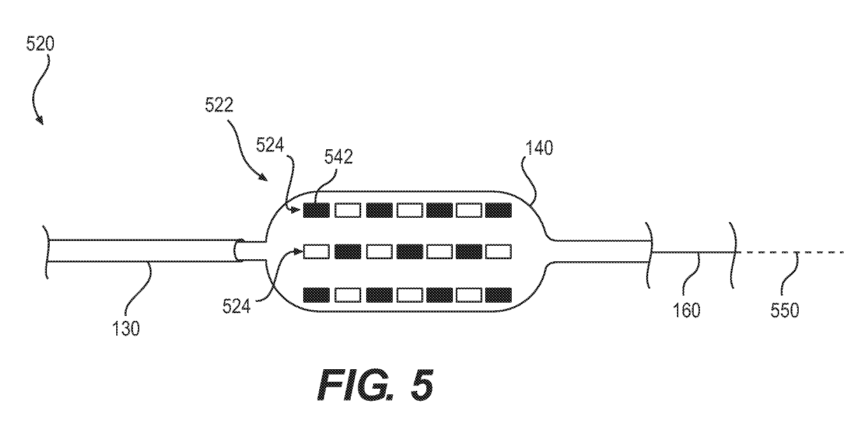

An energy delivery device 520 is shown in FIG. 5. Energy delivery device 520 may be substantially similar to energy delivery device 120 described above, except that energy delivery device 520 may include a matrix 522 of electrodes spaced about the outer surface of expandable member 140. Matrix 522 may be formed by a plurality of rows 524 of electrodes 542. Each of the rows 524 may be circumferentially spaced from adjacent rows 524. Further, each row 524 may include a plurality of electrodes 542 that are longitudinally spaced from one another. A given electrode 542 of matrix 522 may be longitudinally spaced from one or more electrodes, and also may be circumferentially spaced from one or more electrodes.

Each row 524 of electrodes may include the same number of electrodes as one another, and the spacing of electrodes may be uniform within each row 524, and throughout all rows 524. Thus, the spacing between any two adjacent and longitudinally spaced electrodes may be the same as the spacing between any other two adjacent and longitudinally spaced electrodes. Similarly, the spacing between any two adjacent and circumferentially spaced electrodes may be the same as the spacing between any other two adjacent and circumferentially spaced electrodes.

Each row 524 may include electrodes 542 extending from a proximal end of expandable member 140 to a distal end of expandable member 140. Each electrode 542 of a given row 524 may occupy a particular position in a sequence within its respective row 524. For example, the proximalmost electrode of a given row 524 may occupy the first position of a given row. The electrode 542 that is immediately distally adjacent to the proximalmost electrode 542 may occupy the second position of a given row 524, and so forth. The distalmost electrode 542 may occupy an nth position of a given row 524, wherein n may be the number of electrodes within the row 524. As set forth above, the spacing of electrodes 542 may be uniform throughout all rows 524. A given electrode from a row 524 may lie in a plane that is normal to the longitudinal axis 550 of expandable member 140 with correspondingly positioned electrodes from each other row 524. For example, a portion of the proximalmost electrode of each row 524 may lie in a single plane that is normal to longitudinal axis 550. Additionally, a portion of the proximalmost electrode of each row 524 may be disposed at the same distance from any point taken along the central longitudinal axis 550. This relationship among the electrodes 542 of rows 524 may be shared among other groups of correspondingly positioned electrodes. For example, a portion of the second electrode of each row 524 may lie in a single plane that is normal to longitudinal axis 550.

The arrangement of FIG. 5 may provide selective circumferential and longitudinal application of electric fields while energy delivery device 520 is disposed within a body lumen, allowing for the targeted application of energy to tumor tissue that may be disposed in various patterns about the body lumen. The arrangement of FIG. 6 set forth below may provide similar benefits.

FIG. 6 illustrates an example of an energy delivery device 600. The energy delivery device 600 may have a catheter body 602, the distal end of which may include an expandable basket 610 having a plurality of legs 612.

Each of the plurality of legs 612 may be configured to converge toward an atraumatic distal tip 614 of expandable basket 610. Each leg 612 may include a plurality of electrodes 642. The electrodes 642 of a given leg 612 may be longitudinally spaced from one another. Accordingly, the electrodes 642 may include, but are not limited to, band electrodes or dot electrodes formed on the surface of leg 612. Longitudinally spaced and adjacent electrodes on a given leg 612 may be insulated from one another such that the proximal and distal end of each electrode 642 is defined by an electrically non-conductive material.

Each leg 612 may include any number of electrodes. In the example shown by FIG. 6, each leg 612 may include eight longitudinally spaced electrodes 612. Additional or fewer electrodes 642 may be disposed on each leg 612. A lead (not shown) may be electrically coupled to each electrode 642. The leads may extend through the catheter toward controller 110 and/or energy generator 112. Alternatively, it is contemplated that each leg 612 may be formed of a single, elongate electrode. In this alternative example, the elongate electrode may include electrical insulator material covering a proximal portion and/or a distal portion of the elongate electrode.

In some examples, legs 612 may include a resilient inert material, such as, e.g., Nitinol metal or silicone rubber. In the illustrated example of FIG. 6, expandable basket 610 includes four legs 612 that are radially spaced from one another at substantially equal intervals. It is contemplated that any other number of legs 612 may form expandable basket 610, and that legs 612 may be spaced from one another at uneven intervals.

Energy delivery device 600 may include an outer sheath 620 that is movable along the longitudinal axis of the catheter 602. The energy delivery device 600 also may include an actuating member (e.g., a wire) 616 that may be coupled at a proximal end to actuator 116 (referring to FIG. 1). The actuating member 616 may extend through a lumen of catheter 602, through a volume defined by the plurality of legs 612, to the distal tip 604. The plurality of legs 612 may be configured to form an expandable basket-type shape when in a second, expanded configuration. Accordingly, upon expansion of basket 610 from a first, collapsed configuration to the second, expanded configuration, each of the plurality of legs 612 may be configured to bow radially outward away from a longitudinal axis 630 of energy delivery device 600 as wire 616 moves proximally. Expandable basket 610 may further be configured to return to the first, collapsed configuration upon release of wire 616, which may cause each of the plurality of legs 612 to move radially inward toward the longitudinal axis 630.

The expandable basket 610 may be moved between the collapsed configuration and the expanded configuration using other mechanisms. For example, expandable basket 610 may be self-expandable and biased toward the expanded configuration such that moving the sheath 620 forward toward the distal end may cause the sheath 620 to move over the expandable basket 610, thereby collapsing the basket 610 into a collapsed configuration. In contrast, moving the sheath 620 proximally toward the proximal end) may allow the expandable basket 610 to spring open and assume the expanded configuration shown in FIG. 6. Self-expansion may occur because basket legs 612 may be formed with a pre-set configuration from a material capable of being compressed to a generally compressed configuration without plastic deformation. Such materials may include, e.g., shape memory alloys, including, but not limited to, nitinol. As all basket legs 612 restore themselves to the expanded configuration, expandable basket 610 may expand until each leg makes contact with a wall of a body lumen.

Alternatively, actuating member 616 may be a rigid pushing member. In response to a distally applied force, the pushing member may push expandable basket distally out of sheath 620, causing expandable basket 610 to spring radially outward into the second, expanded configuration. Other modes of expansion, such as expansion by employing a balloon device within expandable basket 610, may be employed as desired and such variations are within the ability of those in the art to design and deploy.

Energy delivery device 600 also may include sensors configured to help determine the degree to which the expandable basket 610 expands. For example, expansion may cause the individual basket legs 612 to change shape, and therefore a strain gauge (not shown) could be mounted on one or more basket legs 612. The strain gauge may be configured to measure the degree of strain on, for example, a basket leg, which may correspond to basket expansion. Other methods will be apparent to those of ordinary skill in the art. Controller 110 may utilize the degree of basket expansion to determine the distance between circumferentially spaced electrodes on adjacent basket legs 612. In yet another example, a full circumferential electrode treatment algorithm may be employed without using radial impedance measurements.

Energy Delivery Methods

Controller 110 may be configured to utilize impedance to operate the various energy delivery devices described above. Prior to treatment, impedance values of tissue surrounding a body lumen may be determined in order to identify potential treatment locations. In one example, the impedance of tissue disposed between circumferentially- and/or longitudinally-spaced adjacent electrodes may be measured in order to create a virtual map of tissue surrounding an energy delivery device. The virtual map may be used to differentiate between healthy tissue and cancerous tissue (e.g., tumors) in order to selectively treat cancerous tissue and reduce the amount of healthy tissue that is damaged.

In some examples, an operator may review the impedance measurements and manually determine which electrodes of a given energy delivery device to activate. In other examples, controller 110 may analyze the impedance measurements and automatically determine a treatment pattern based on the measurements. In such examples, controller 110 may compare the impedance results to various thresholds to determine whether a particular region of tissue surrounding the energy delivery device is cancerous or healthy. Normally cancerous tissue is more conductive than normal tissue and thus may have a smaller impedance value. The exact value may depend on multiple factors such as frequency, electrode size, electrode spacing, electrode shape, cancer type and tissue type. In one example, healthy tissue may have an impedance value around 200 ohms, and cancerous tissue may have an impedance value around 120 ohms, and a threshold value may be set at 140 ohms. In some examples, controller 110 may use closed loop measurements and control algorithms to assist in targeted ablation treatment across electrode pairs.

Prior to treatment, controller 110 may send test pulses between each pair of adjacent electrodes (both circumferentially adjacent electrodes and longitudinally adjacent electrodes) in order to create a detailed map of the tissue surrounding the energy delivery device. For example, FIG. 7 illustrates an energy delivery device disposed in an expanded configuration within a body lumen. The body lumen may be surrounded by healthy tissue 702 and cancerous tissue 704. Controller 110 may send test pulses between each pair of circumferentially adjacent electrodes (142a-144a, 144a-142b, 142b-144b, 144b-142c, 142c-144c, 144c-142d, 142d-144d, 144d-142a) to determine whether cancerous tissue is disposed between any given pair of electrodes. In the example of FIG. 7, once the impedance measurements are collected, controller 110 may determine based on an analysis of those measurements, that the measured impedance between electrode pairs 142a-144a, 144a-142b, 142b-144b, 144b-142c, and 142c-144c indicates that cancerous tissue is disposed between those pairs. For example, the impedance measured between the pairs 142a-144a, 144a-142b, 142b-144b, 144b-142c, and 142c-144c may be below an impedance threshold indicative of cancerous tissue between those electrode pairs. Controller 110 or an operator then may utilize that determination to selectively deliver electroporation therapy between electrode pairs 142a-144a, 144a-142b, 142b-144b, 144b-142c, and 142c-144c. In some examples, the impedance measured between electrode pairs 142a-144a and 142c-144c may be less than the impedance measured between electrode pairs 144a-142b, 142b-144b, and 144b-142c, because the tissue between electrode pairs 142a-144a and 142c-144c may include both healthy tissue and cancerous tissue. However, controller 110 may nonetheless apply electroporation therapy between electrode pairs 142a-144a and 142c-144c to ensure that as much cancerous tissue as possible is treated.

Controller 110 may apply a similar methodology when determining whether to deliver therapy between longitudinally adjacent electrodes. Referring to FIG. 5, a given row 524 of electrodes is shown having seven electrodes. If cancerous tissue is detected between the first and fifth electrodes, but not between the fifth and seventh electrodes, therapy will be applied only between adjacent electrodes in the group of electrodes spanning from the first electrode to the fifth electrode (e.g., 1-2, 2-3, 3-4, and 4-5). In this example, therapy would not be applied between the fifth and sixth electrodes and the sixth and seventh electrodes.

For electroporation, high voltage pulses may be used to create transient pores within cells exposed to the electric field, allowing the cells to be loaded with a therapeutic agent (e.g., due to diffusion, migration or both). Electroporation also may create permanent pores that lead to cell death within targeted tissues. The density and size of the pores of the cell membrane depend, for example, on the electric field parameters and polarity. In some example, irreversible electroporation and cell death may require that an electric field intensity of at least 1000 V/cm be achieved, although other suitable electric field intensities may also be utilized. For irreversible electroporation, a lower field density (e.g. 500 V/cm) may cause only fifty percent cell destruction or a population of tumor cells to survive. The field density may be affected by electrode spacing (e.g., 1-10 mm), applied voltage (e.g., 100-500V), and permittivity of the tumor or cell membrane. Electroporation therapy may be coupled with other vascular disrupting agents (VDAs) or solvents (e.g., DMSO, ethanol) that affect the membrane impedance. When used in conjunction with these other therapies, lower voltages may be applied to create a similar therapeutic effect achieved by higher voltages. Energy delivery devices utilizing an expandable basket may be particularly suited for use in, e.g., the lung airways, where occlusion of the airway may be undesirable. On the other hand, energy delivery devices utilizing a balloon may be particularly suited for use in body lumens having significant fluid disposed therein.

As set forth above, therapeutic agents may be introduced to cancerous tissues before, during, or after electroporation therapy is applied. The agent may be introduced by direct drug injection, by systemic drug introduction, such as chemical therapy, or by local drug perfusion. Delivery could also be achieved by a balloon (porous or weeping), needle injection (via array), or a bolus of biodegradable microparticles or beads. The agents may be delivered shortly after application while the cellular pores are still viable and opened (for example, within one to five minutes). One protocol may be a series of administered physical ablations followed by a chemical introduction then re-ablation using IRE to promote a mass diffusion gradient into the cell membrane.

Exemplary therapeutic agents that may help prevent excessive cell growth may include Paclitaxel, and various olimus drugs (everolimus, sirolimus). The term “therapeutic agent” as used in the present disclosure may encompass therapeutic agents, genetic materials, and biological materials and can be used interchangeably with “biologically active material.” In one example, the therapeutic agent may be an anti-cell proliferation (restenotic) agent. In other examples, the therapeutic agent may inhibit smooth muscle contraction, migration or hyperactivity, mucus production and mucus thickening.

In some examples, IRE treatments disclosed herein may also spur an immune response as cellular proteins are ejected from the cell membrane after nanopores are created causing a trigger of T-cell recognition and response.

The disclosed energy delivery devices may be used to treat cancerous tissue in any suitable body lumen, such as, e.g., bile ducts, pancreatic lumens, liver lumens, biliary ducts, bladder or stomach pouches, intestines, the colon, renal arteries, and airways of the lung, among other body lumens.

Examples of the present disclosure may damage cancerous tissues via non-thermal lesions so as to avoid tissue charring. Electroporation therapies also may overcome the heat sink effect of blood or fluid flow, which can reduce the effectiveness of thermal ablation treatments. Electroporation also may spare structure protein such as nerves, vessels, bile ducts, and the like, and may not cause damage to the luminal structure.

Other examples of the present disclosure will be apparent to those skilled in the art from consideration of the specification and practice of the present disclosure disclosed herein. It is intended that the specification and examples be considered as exemplary only, with a true scope and spirit of the present disclosure being indicated by the following claims.

What is claimed is:

1. An energy delivery system, comprising:
an expandable member having a plurality of electrodes, including pairs of adjacent electrodes;
a generator configured to supply an electric voltage to each of the plurality of electrodes; and
a controller coupled to the plurality of electrodes and the generator, wherein the controller is configured to:
measure an impedance of tissue disposed between the electrodes of each pair of adjacent electrodes;
determine, based on the measured impedances, whether cancerous tissue is disposed between any of the electrodes of each pair of adjacent electrodes; and
apply a voltage between the electrodes of each pair of adjacent electrodes determined to have cancerous tissue disposed between them.

2. The energy delivery device of claim 1, wherein each electrode of the plurality of electrodes is circumferentially adjacent to one or more electrodes and longitudinally adjacent to one or more electrodes

3. The energy delivery system of claim 1, wherein the expandable member includes a plurality of rows of electrodes, wherein each row of electrodes includes a plurality of longitudinally spaced electrodes, wherein the plurality of rows are circumferentially spaced from one another.

4. The energy delivery system of claim 1, wherein all adjacent and circumferentially spaced electrodes are spaced apart from one another by the same circumferential distance.

5. The energy delivery system of claim 1, wherein all adjacent and longitudinally spaced electrodes are spaced apart from one another by the same longitudinal distance.

6. The energy delivery system of claim 1, wherein the expandable member is a balloon, wherein the plurality of electrodes are disposed on or adjacent to an outer surface of the balloon.

7. The energy delivery system of claim 1, wherein at least some of the plurality of electrodes include flexible circuits attached to an outer surface of the expandable member.

8. The energy delivery system of claim 1, wherein the expandable member is an expandable basket having a plurality of legs, wherein each leg of the plurality of legs includes a plurality of longitudinally spaced electrodes.

9. The energy delivery system of claim 8, further including an actuating member that extends from a proximal end of the expandable member, through a volume defined by the plurality of legs, to a distal end of the expandable member.

10. The energy delivery system of claim 1, wherein the plurality of electrodes are coupled to opposite poles of the generator.

11. The energy delivery system of claim 10, wherein adjacent electrodes of the plurality of electrodes are configured to deliver energy of opposite polarity.

12. The energy delivery system of claim 1, wherein the controller determines that cancerous tissue is located between a pair of circumferentially adjacent electrodes or a pair of longitudinally adjacent electrodes if the measured impedance between the pair of circumferentially adjacent electrodes or the pair of longitudinally adjacent electrodes is smaller than a threshold impedance value.

13. The energy delivery system of claim 1, wherein the generator is configured to supply voltage to each of the electrodes in a manner sufficient to cause electroporation of tissue.

14. The energy delivery system of any claim 1, wherein the generator is configured to create an electrical field strength within tissue from 250 to 3000 volt/cm.

15. The energy delivery system of claim 1, wherein the generator is configured to deliver pulses with a pulse width from 1 ns to 1000 μs and a pulse interval from 10 ms to 100 s.

16. An energy delivery system, comprising:
an expandable balloon;
a plurality of electrodes disposed on an outer surface of the balloon, wherein each electrode of the plurality of electrodes is circumferentially adjacent to one or more electrodes and longitudinally adjacent to one or more electrodes;
a generator configured to supply an electric voltage to each of the plurality of electrodes, wherein any two circumferentially adjacent electrodes are coupled to opposite poles of the generator, and wherein any two longitudinally adjacent electrodes are coupled to opposite poles of the generator, wherein the generator is configured to supply the electric voltage in a manner sufficient to cause electroporation of tissue; and
a controller coupled to the plurality of electrodes and the generator, wherein the controller is configured to:
measure an impedance of tissue disposed between pairs of circumferentially adjacent electrodes and pairs of longitudinally adjacent electrodes;
determine, based on the measured impedances, whether cancerous tissue is disposed between any pairs of circumferentially adjacent electrodes and any pairs of longitudinally adjacent electrodes; and
apply a voltage only between any pairs of circumferentially adjacent electrodes and any pairs of longitudinally adjacent electrodes determined to have cancerous tissues disposed between them, wherein the controller determines that cancerous tissue is located between a pair of circumferentially adjacent electrodes or a pair of longitudinally adjacent electrodes if the measured impedance between the pair of circumferentially adjacent electrodes or the pair of longitudinally adjacent electrodes is smaller than a threshold impedance value.

17. The energy delivery system of claim 16, wherein the expandable member includes a plurality of rows of electrodes, wherein each row of electrodes includes a plurality of longitudinally spaced electrodes, wherein each electrode of the plurality of electrodes is positioned within one of the plurality of rows, and lies in a single plane normal to a longitudinal axis of the expandable member with a correspondingly positioned electrode from each other row.

18. A method of treating tissue adjacent a body lumen, the method comprising:
measuring an impedance of tissue disposed between pairs of adjacent electrodes of an energy delivery device;
applying an electric field only between pairs of adjacent electrodes determined to have cancerous tissue disposed between them, wherein a controller determines that cancerous tissue is disposed between a given pair of adjacent electrodes based on the measured impedance between the given pair of adjacent electrodes, wherein the electric field causes electroporation of the tissue disposed within the electric field.

19. The method of claim 18, wherein the body lumen is a bile duct, a lung airway, a hepatic artery, or a lumen of a liver or pancreas.

20. The method of claim 18, wherein the electric field is a bipolar electric field.
